(19) United States
(12) Patent Application Publication
(10) Pub. No.: US 2015/0320587 A1
(43) Pub. Date: Nov. 12, 2015

(54) VAGINAL PROTECTOR

(71) Applicant: Olive Coker, Stockbridge, GA (US)

(72) Inventor: Olive Coker, Stockbridge, GA (US)

(21) Appl. No.: 14/273,668

(22) Filed: May 9, 2014

(51) Int. Cl. A61F 6/06

(52) U.S. Cl. CPC A61F 6/06

(57) ABSTRACT

A vaginal protector for protecting the genital area of a female including: a surface shield, where the surface shield includes an inside surface and an outside surface; an adhesive applied to the inside surface; a stub extending from the inside surface; a vaginal plug secured over the stub for insertion into a female's vagina during use; and an opening above the stub, where the opening allows for any flow of urine from the female. The plug may extend from the surface shield at a perpendicular angle and preferably the inside surface covers a labia area of the female.

### BACKGROUND OF THE INVENTION

1. Field of Invention

The field of invention relates to a vaginal protector that protects the vaginal region of a bedridden female.

2. Description of Related Art

Many bedridden females in particular elderly female patients encounter difficulties in maintaining a clean vaginal area during their time in the bed. Many times these females have adult diapers in place to receive any waste that are expelled while in the bed. As a consequence, fecal matter may make its way into the vaginal area causing major problems for the female patient. It's usually very difficult and time-consuming to remove the fecal matter from the vagina. This task may be further exacerbated if the fecal matter is runny or if the patient has a femoral compaction or any type of joint disease or injury. Further the fecal matter usually carries bacteria that may then lead to vaginal infections. Further depending on the volume of the fecal matter or bowel movement the urethra orifice may also take bacteria into the bladder causing bladder and urinary tract infections. As a consequence, it would therefore be desirable to have a device or mechanism that can protect the vaginal area from fecal matter and other bacteria of a bedridden female. Ideally the device may be inserted into the vagina and provide adequate protection of the vagina surrounding skin surface areas.

### SUMMARY OF THE INVENTION

The present invention relates to a vaginal protector for protecting the genital area of a female including: a surface shield, where the surface shield includes an inside surface and an outside surface; an adhesive applied to the inside surface; a stub extending from the inside surface; a vaginal plug secured over the stub for insertion into a female's vagina during use; and an opening above the stub, where the opening allows for any flow of urine from the female. The plug may extend from the surface shield at a perpendicular angle and preferably the inside surface covers a labia area of the female.

### BRIEF DESCRIPTION OF DRAWINGS

FIG. 1A depicts a prospective view of a vaginal shield employed in accordance with the present invention.

FIG. 1B depicts an exploded view of the vaginal shield in accordance with the present invention.

FIG. 2A shows a front surface of the vaginal shield according to the present invention.

FIG. 2B depicts the side view of the vaginal shield and plug in accordance with the present invention.

FIG. 3 depicts the top view of the vaginal shield and plug in accordance with the present invention.

FIG. 4A depicts use the vaginal shield and plug in accordance with the present invention.

FIG. 4B depicts removal of the vaginal shield and plug from a vagina.

### DETAILED DESCRIPTION

The present invention relates to a vaginal shield and plug that is used to protect the vagina and surrounding areas from bacteria which is especially designed for a bedridden female. The vaginal shield and plug according to the present invention is a plug and a stem that extends from the shield where the shield surrounds the labia area and the plug inserts into the vagina of a patient. Adhesive is used on the inside surface of the shield to adhere the shield and plug into a stationary position to avoid movement. Once inserted this shield and plug protects the vagina from any bacteria that may be existing within any fecal matter that is expelled by the patient. The surface shield also includes an opening that allows for the expulsion of urine through the opening where the opening exposes the area surrounding the clitoris. In general the shape of the surface shield resembles the shape of the female genitalia area, where the shield flares outwards in a triangular shape to cover the labia and then opening is provided that allows for the expulsion of urine near the clitoris of the female.

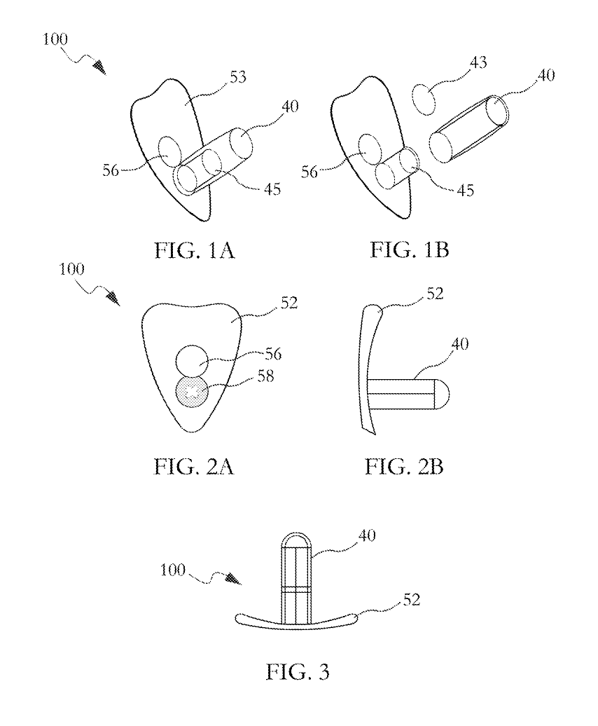

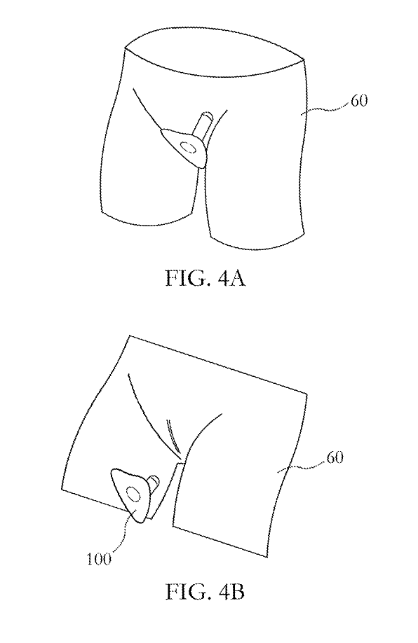

In reference to FIG. 1A, a prospective view of a vaginal shield 100 is depicted. Shown in FIG. 1A is an inside surface 53 of a surface shield 52. An opening 56 is shown above a plug 40 that extends from the inside surface 53. The plug 40 inserts into a vagina of the patient as the shield's inside surface 53 covers a labia area of the patient. An exploded view is shown in FIG. 1B. In this exploded view a stub 45 is shown that allows for the placement of the plug 40 over the stub 45 that extends from the inside surface 53. A valve 43 is also shown that inserts into the opening 56. The valve 43 allows for the transfer or expulsion of urine from the female but provides adequate protection from any fecal matter or other bacteria that may be on the outside of the surface shield 52. A front view of the surface shield 52 is shown in FIG. 2A which clearly shows the opening 56. Below the opening 56 is a base 58 of the stub 45. FIG. 2B shows the side view of the vaginal shield 100. As shown the plug 40 extends in a perpendicular angle from the surface shield 52. A top view of this arrangement is also depicted in FIG. 3.

Exemplary use of the vaginal shield 100 is shown in FIG. 4A. As shown the vaginal shield 100 is inserted into a vagina of a user 60. The shield 100 covers the labia area providing protection for the user 60. FIG. 4B depicts the shield 100 removed from the user 60 and exposure of the labia area of the user 60.

During use this vaginal shield provides a cover of the female genitalia protecting it from bacteria and fecal matter. In the preferred embodiment, the plug is made of a soft pliable material that essentially mimics the shape of the vagina once it is inserted. The plug may be also made of a non-permeable material so that no liquid or bacteria can enter. The surface shield 52 covers both the labia major and minor where the clitoris is positioned near the opening that is covered with a valve so that urine may be expelled during use of the vaginal shield. Further the vaginal shield is preferable made from hypoallergenic material to avoid skin irritation and breakdown.

An adhesive is placed on the inside surface 53 to adhere the shield onto the skin of the patient and one particular embodiment nano-polymers may be used as an effective adhesive to easily adhere and remove the vaginal shield. Use of the vaginal shield and plug prevents infection and spread of bacteria into the vaginal and female genitalia area. Further the shield may be produced in various sizes to accommodate various sizes of vaginal openings and the size of female genitalia such as the labia areas. The instant invention has been shown and described in what it considers to be the most practical and preferred embodiments. It is recognized, however, that departures may be made there from within the scope of the invention and that obvious modifications will occur to a person skilled in the art.

What is claimed is:

1. A vaginal protector for protecting the genital area of a female comprising:
a. a surface shield, where the surface shield includes an inside surface and an outside surface;
b. an adhesive applied to the inside surface;
c. a stub extending from the inside surface;
d. a vaginal plug secured over the stub for insertion into a female's vagina during use; and
e. an opening above the stub, where the opening allows for any flow of urine from the female.

2. The vaginal protector according to claim 1, wherein the plug extends from the surface shield at a perpendicular angle.

3. The vaginal protector according to claim 1, wherein the inside surface covers a labia area of the female.

4. The vaginal protector according to claim 1, wherein the plug is made of a soft pliable material.

5. The vaginal protector according to claim 1, wherein the plug is made of a non-permeable material.

6. The vaginal protector according to claim 1, where the adhesive includes nano-polymers.

7. The vaginal protector according to claim 3, where the inside surface covers both the labia major and labia minor.

8. The vaginal protector according to claim 1, where the opening is positioned over the clitoris of the female.

9. The vaginal protector according to claim 1, where the surface shield is made of hypoallergenic materials.
